(19) United States
(12) Patent Application Publication
(10) Pub. No.: US 2014/0236063 A1
(43) Pub. Date: Aug. 21, 2014

(54) ARTERIOVENOUS ACCESS VALVE SYSTEM AND PROCESS

(71) Applicant: David L. Cull, Greenville, SC (US)

(72) Inventor: David L. Cull, Greenville, SC (US)

(73) Assignee: CREATIVASC MEDICAL, LLC, Greenville, SC (US)

(21) Appl. No.: 14/346,341

(22) PCT Filed: Sep. 21, 2012
(86) PCT No.: PCT/US2012/056611

§ 371 Date: Mar. 21, 2014

Related U.S. Application Data
(60) Provisional application No. 61/538,394, filed on Sep. 23, 2011.

(51) Int. Cl. A61M 1/36; A61F 2/82

(52) U.S. Cl. CPC A61M 1/3655; A61F 2/82; A61M 2205/3331
USPC 604/6.16; 623/1.24

(57) ABSTRACT

In accordance with certain aspects of the present disclosure, a subcutaneous arteriovenous graft system is described. The system includes an arteriovenous graft having an arterial end and an opposite venous end, at least one of the arterial end and venous end including a support frame. The system further includes a first valve device positioned at the arterial end of the arteriovenous graft and a second valve device positioned at the venous end of the arteriovenous graft and an actuator in communication with both the first valve device and the second valve device, the actuator being configured to cause each valve device to open or close simultaneously.

### BACKGROUND

The function of kidneys, which are glandular organs located in the upper abdominal cavity of vertebrates, is to filter blood and remove waste products. Specifically, kidneys separate water and waste products of metabolism from blood and excrete them as urine through the bladder. Chronic renal failure is a disease of the kidney in which the kidney function breaks down and is no longer able to filter blood and remove waste substances. Should certain toxic waste substances not be removed from the blood, the toxic substances may increase to lethal concentrations within the body.

Hemodialysis is a life-sustaining treatment for patients who have renal failure. Hemodialysis is a process whereby the patient's blood is filtered and toxins are removed using an extracorporeal dialysis machine. For hemodialysis to be effective, large volumes of blood must be removed rapidly from the patient's body, passed through the dialysis machine, and returned to the patient. A number of operations have been developed to provide access to the circulation system of a patient such that patients may be connected to the dialysis machine.

For example, the most commonly performed hemodialysis access operation is a subcutaneous placement of an arteriovenous graft, which is made from a biocompatible tube. The biocompatible tube can be made of, for instance, a fluoropolymer such as polytetrafluoroethylene. One end of the tube is connected to an artery while the other end is connected to a vein. The arteriovenous graft is typically placed either in the leg or arm of a patient.

Blood flows from the artery, through the graft and into the vein. To connect the patient to a dialysis machine, two large hypodermic needles are inserted through the skin and into the graft. Blood is removed from the patient through one needle, circulated through the dialysis machine, and returned to the patient through the second needle. Typically, patients undergo hemodialysis approximately four hours a day, three days a week.

Various problems, however, have been experienced with the use of an arteriovenous graft. For example, arterial steal occurs when excessive blood flow through the arteriovenous graft “steals” blood from the distal arterial bed. Arterial steal can prevent the proper supply of blood from reaching the extremity of a patient.

To address such problems, systems and processes have been deployed which can minimize or prevent complications by closing the arteriovenous graft when hemodialysis is not taking place. An example of one such system is described in U.S. Pat. No. 7,025,741 entitled “Arteriovenous access valve system and process”, incorporated by reference herein. These systems and processes can utilize valves, such as balloon valves, to force closure of one or more portions of an arteriovenous graft by pressing the arteriovenous graft walls together.

However, after being closed tightly for a period of time, existing arteriovenous grafts have a tendency to remain closed due in part to the “memory” of conventional biocompatible materials as well as remaining blood cells between the walls of the graft that can act to adhere the walls together.

In view of the above, a need exists in the art for an arteriovenous graft that can prevent and minimize arterial steal and graft thrombosis. A process for using an arteriovenous graft in minimizing arterial steal and graft thrombosis is also needed. More particularly, a need exists for an arteriovenous graft that can close tightly under pressure and also reopen when the pressure is removed.

### SUMMARY

In accordance with certain aspects of the present disclosure, a subcutaneous arteriovenous graft system is described. The system includes an arteriovenous graft having an arterial end and an opposite venous end, at least one of the arterial end and venous end including a support frame. The system further includes a first valve device positioned at the arterial end of the arteriovenous graft and a second valve device positioned at the venous end of the arteriovenous graft and an actuator in communication with both the first valve device and the second valve device, the actuator being configured to cause each valve device to open or close simultaneously.

In still other embodiments of the present disclosure, a hemodialysis method is described. An arteriovenous graft system is implanted in a patient. The arteriovenous graft system includes an arteriovenous graft having a first end that is connected to an artery and a second end that is connected to a vein, at least one of the arterial end and venous end including a support frame. The arteriovenous graft system further includes a first valve device positioned at the arterial end of the arteriovenous graft and a second valve device positioned at the venous end of the arteriovenous graft. An actuator is in communication with both the first valve device and the second valve device. The first and second valve devices are opened simultaneously using the actuator causing blood to flow through the arteriovenous graft. First and second hypodermic needles are inserted into the arteriovenous graft, the hypodermic needles being in fluid communication with a hemodialysis machine. Blood circulates through the hemodialysis machine and after a sufficient amount of blood has been circulated through the hemodialysis machine, the first and second valve devices are closed using the actuator.

Other features and aspects of the present disclosure are discussed in greater detail below.

### BRIEF DESCRIPTION OF THE DRAWINGS

A full and enabling disclosure of the present invention is set forth in the specification with reference to the following figures.

FIG. 1 is a side view with cut away portions of a human arm illustrating the placement of an arteriovenous graft;

FIG. 2 is a perspective view of embodiments of arteriovenous graft systems made in accordance with the present disclosure; and

FIG. 3 is a top view of embodiments of arteriovenous graft systems made in accordance with the present disclosure,

Repeat use of reference characters in the present specification and drawings is intended to represent the same or analogous features of the disclosure.

### DETAILED DESCRIPTION

Reference now will be made to the embodiments of the disclosure, one or more examples of which are set forth below. Each example is provided by way of explanation of the disclosure, not as a limitation of the disclosure. In fact, it will be apparent to those skilled in the art that various modifications and variations may be made in the disclosure without departing from the scope or spirit of the disclosure. For instance, features illustrated or described as part of one embodiment may be used in another embodiment to yield a still further embodiment. For example, an arteriovenous graft system may include combinations of the valve devices described below. Thus, it is intended that the present disclosure cover such modifications and variations as come within the scope of the appended claims and their equivalents. It is to be understood by one of ordinary skill in the art that the present discussion is a description of exemplary embodiments only, and is not intended as limiting the broader aspects of the present disclosure, which broader aspects are embodied in the exemplary construction.

In general, the present disclosure is directed to an implantable arteriovenous graft system that may be used in carrying out hemodialysis treatments, In particular, the arteriovenous graft system includes one or more support frames to allow the arteriovenous graft to readily reopen for hemodialysis treatment after a period of closure. Although the following description will refer to the arteriovenous graft system being implanted into an arm, it should be understood that the system may be implanted in any suitable location of the body. For example, in other embodiments, the arteriovenous graft system may be implanted into a leg.

In addition to being well suited for carrying out hemodialysis, the arteriovenous graft system of the present disclosure also prevents or minimizes arterial steal and graft thrombosis. In particular, the arteriovenous graft system is designed to prevent or minimize blood flow through the graft when hemodialysis is not occurring.

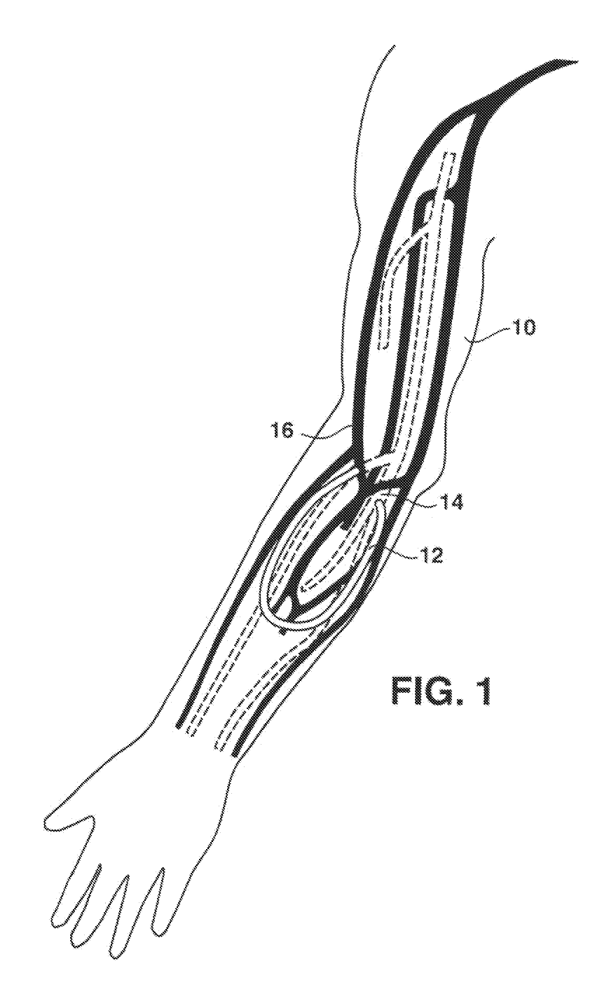

Referring to FIG. 1, for purposes of explanation, a right arm 10 of a patient is shown. Selected arteries (shown as dotted pathways) are illustrated in conjunction with selected veins (shown as dark pathways). An arteriovenous graft 12 is shown connected at one end to an artery and at an opposite end to a vein, In particular, the arteriovenous graft 12 is connected to the brachial artery 14 and to the cephalic vein 16.

The arteriovenous graft 12 is made from any suitable biocompatible material. For example, in one embodiment, the graft is made from a fluoropolymer, such as polytetrafluoroethylene, which is commercially available as GORTEX™ from the W. L. Gore Company.

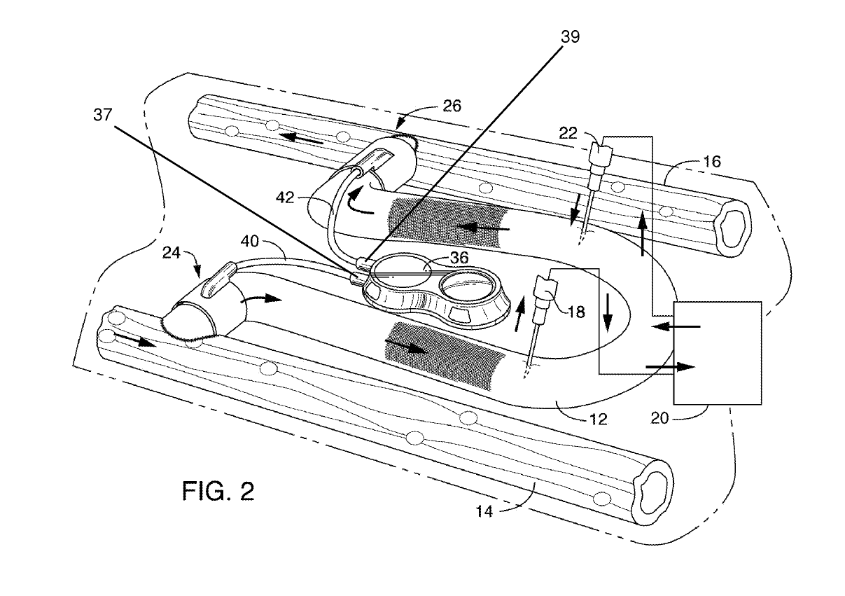

Referring to FIG. 2, one embodiment of an arteriovenous graft system made in accordance with the present disclosure is shown including an arteriovenous graft 12. As illustrated, the arteriovenous graft 12 is connected to an artery 14 and to a vein 16. In order to carry out hemodialysis, a first hypodermic needle 18 is inserted through the skin and into the arteriovenous graft 12. Blood is removed from the arteriovenous graft 12 through the needle and into a dialysis machine 20. In the dialysis machine, waste materials are removed from the blood. After circulating through the dialysis machine 20, the blood is then fed back into the arteriovenous graft 12 through a second hypodermic needle 22.

In accordance with the present disclosure, the arteriovenous graft system as shown in FIG. 2 includes at least a first valve device generally 24 positioned at the arterial end of the arteriovenous graft 12. Exemplary arteriovenous graft systems are described in U.S. patent application Ser. Nos. 11/364,801, 11/807,479, and 12/202,664, all incorporated by reference herein. Optionally, the arteriovenous graft system can further include a second valve device generally 26 positioned at the venous end of the arteriovenous graft. In this regard, one or more of the valve devices 24, 26 can also have a complimentary shape to the artery and/or vein and define holes (not shown) to permit direct suturing between the device(s) and the artery and/or vein to further reinforce the connection and prevent the valve device from moving away from its intended location. The valve devices 24 and 26 are in an open position during normal hemodialysis as shown in FIG. 2. When hemodialysis has ended, however, the valve devices 24 and 26 are moved to a closed position in order to prevent blood flow through the arteriovenous graft. In this manner, arterial steal is either eliminated or reduced. Further, by reducing turbulent blood flow through the arteriovenous graft, graft thrombosis is also prevented.

In addition to minimizing arterial steal and preventing graft thrombosis, the system and the process of the present disclosure also offer various other advantages. For example, reducing or stopping blood flow through the arteriovenous graft when hemodialysis is not occurring also prevents the graft from bleeding when the hypodermic needles used to carry out hemodialysis are removed from the graft. Hypodermic needles, for instance, usually have a relatively large diameter or gauge. Thus, when the needles are removed from a graft, bleeding can occur where the needles have previously been, Needle hole bleeding through the graft can result in the formation of scar tissue and graft pseudoaneurisms. These complications, however, may be prevented through the use of the system of the present disclosure.

In the embodiment shown in FIG. 2, the valve devices 24 and 26 each include an inflatable balloon (not shown). When inflated, the balloons constrict the arteriovenous graft 12 for reducing or eliminating blood flow through the graft. The first valve device 24 and second valve device 26 are both connected to actuator 200. Actuator 36 is configured to open or close both valve devices simultaneously. For example, in the illustrated embodiment, actuator 36 is in fluid communication with first valve device 24 via tubing 40 connected to first outlet nozzle 37 and is in fluid communication with the second valve device 26 via tubing 42 connected to second outlet nozzle 39. Actuator 36 can be configured to be subcutaneously implanted in a patient.

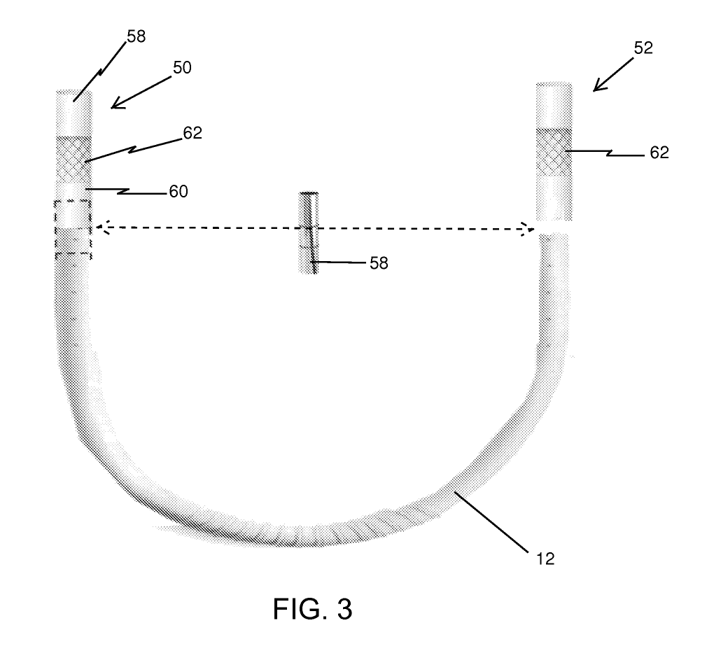

In accordance with the present disclosure, the arteriovenous graft system includes one or more tubular support devices to allow the arteriovenous graft to readily reopen for hemodialysis treatment after a period of closure. Turning to FIG. 3, an arteriovenous graft 12 is illustrated having tubular support devices 50, 52 at the arterial end 54 and venous end 56, respectively thereof. The tubular support devices 50, 52 can be integral to arteriovenous graft 12 as part of the arteriovenous graft or can be separately joined to arteriovenous graft 12 by any suitable mechanism. For instance, as illustrated, coupling insert 58 can be utilized to join one or both tubular support devices 50, 52 to opposite ends of a section of arteriovenous graft tubing. Other suitable mechanisms such a coupler mechanism, conventional suturing, or the like can be used to join one or more join tubular support devices to the arteriovenous graft, either in combination with a coupling insert or other mechanisms or completely separate from any other mechanism. Coupling insert can have a length of from about 0.1 to about 1.0 cm, more particularly from about 0.25 to about 0.75 cm. In this manner, different lengths of arteriovenous graft tubing can be easily utilized depending on the size of tubing needed for a particular patient.

With respect to the tubular support device, again it is contemplated that such device can either be integral to an existing arteriovenous graft or can be joined thereto. Each tubular support device can include one or more support frames 62 that permit repeated opening and closing of the arterial and/or venous section of the arteriovenous graft when acted upon by an aforementioned valve device. A suitable support frame can be a tubular support frame in the form of a conventional stent. Any suitable stent can be utilized including metal stents, Nitinol stents, or the like. The support frame(s) increases radial strength of the device while also promoting recovery. Frame can have a length from about 0.5 to 2.5 cm, more particularly from about 1.0 to 2.0 cm.

In this regard, the support frame can be encapsulated in whole or in part by one or more materials to prevent leakage of blood flow through the arteriovenous graft. For instance, suitable biocompatible materials can be utilized to encapsulate the support frame. The material(s) utilized to encapsulate the support frame can also prevent the surface of the support frame from damaging the valve device.

For example, in certain embodiments, a conventional arteriovenous graft can be utilized to incorporate one or more support frames therein at the opposite ends to form the tubular support device(s). In still other embodiments, a Nitinol stent can be encapsulated such that there is an inner diameter of expanded polytetrafluoroethylene and an outer diameter of expanded polytetrafluoroethylene with the stent sandwiched therebetween. In certain embodiments, a layer of polyurethane can be disposed between the inner and outer diameters of expanded polytetrafluoroethylene to adhere the inner and outer diameters together and prevent delamination while also preventing fluid permeability through the support device. The polyurethane layer can also assure that the sides of the inner diameter close tightly together and prevent the expanded polytetrafluoroethylene from being abraded by the stent. The polyurethane also spreads the forces of suture tension (to the extent sutures are utilized) to join the tubular support device at the anastomosis. However, it should be understood that the materials described are exemplary and that any suitable materials are contemplated for use in connection with the present disclosure.

Turning again to FIG. 3, the tubular support device 50 can include extension portions 58, 60. Extension portion 58 can be utilized to join the tubular support device and the arteriovenous graft to artery at an anastomosis. The anastomosis can be created by any conventional methods such as suturing, stapling, or the like. Referring again to extension portion 58, in certain embodiments tubular support frame 62 does not extend into extension portion 58 and thereby does not interfere with the anastomosis. Similarly, in embodiments in which tubular support device is not integrally connected to arteriovenous graft, extension portion 60 can be utilized to join tubular support device to arteriovenous graft. Again, in certain embodiments tubular support frame 62 does not extend into extension portion 60 and thereby does not interfere with such connection. Extension portions 58, 60 can each have a length of from about 0.5 to 2.5 cm, more particularly from about 1.0 to 2.0 cm.

The process of implanting the system of the present disclosure involves placing a valve device at one or both ends of the arteriovenous graft at the section of the tubular support device (if the tubular support device(s) are not integral with the arteriovenous graft then they are first joined as has previously been described herein to the arteriovenous graft). The valve device(s) is positioned so as to apply pressure to the frame device portion of the respective tubular support device for which it is adjacent to. The arteriovenous graft, including the tubular support device(s), are joined at the anastomosis' as described herein, as is the valve device(s).

It has been determined that blood flowing through the arteriovenous graft has a pressure of from about 100-150 mm Hg so the valve device is designed to exert a pressure of between about 20 psi and 40 psi against the tubular support device. In certain embodiments, the inner diameter of the tubular support device can be pressed together to form a seal at pressure of from about 20 psi to about 25 psi. The systems and processes described herein permit reliable and repeatable open and closing of the tubular support device.

These and other modifications and variations to the present disclosure may be practiced by those of ordinary skill in the art, without departing from the spirit and scope of the present disclosure, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the disclosure so further described in such appended claims.

What is claimed:

1. A subcutaneous arteriovenous graft system comprising:
an arteriovenous graft having an arterial end and an opposite venous end, at least one of the arterial end and venous end comprising a support frame;
a first valve device positioned at the arterial end of the arteriovenous graft and a second valve device positioned at the venous end of the arteriovenous graft; and
an actuator in communication with both the first valve device and the second valve device, the actuator being configured to cause each valve device to open or close simultaneously.

2. A graft system as in claim 1, wherein both the arterial end and the venous end each comprise a support device.

3. A graft system as in claim 1, wherein the support frame comprises a stent.

4. A graft system as in claim 1, wherein the support frame is encapsulated within the graft.

5. A graft system as in claim 1, wherein each valve device is configured to exert a pressure of between about 20 psi and about 40 psi against the support frame.

6. A graft system as in claim 1, further comprising a coupling insert, the coupling insert joining the arteriovenous graft to the support frame.

7. A graft system as in claim 6, wherein the arteriovenous graft and the support frame define a flow path, the coupling insert being positioned within the flow path.

8. A graft system as in claim 1, wherein the arteriovenous graft and the support frame define a flow path, the valve device being configured to obstruct the flow path when closed by the actuator, the support frame being configured to assist in reopening the flow path when the valve device is opened by the actuator.

9. A hemodialysis method comprising:
subcutaneously implanting an arteriovenous graft system in a patient, the arteriovenous graft system including an arteriovenous graft having a first end that is connected to an artery and a second end that is connected to a vein, at least one of the arterial end and venous end comprising a support frame, the arteriovenous graft system further including a first valve device positioned at the arterial end of the arteriovenous graft and a second valve device positioned at the venous end of the arteriovenous graft, the arteriovenous graft system further including an actuator in communication with both the first valve device and the second valve device;
opening the first and second valve devices simultaneously using the actuator causing blood to flow through the arteriovenous graft;
inserting first and second hypodermic needles into the arteriovenous graft, the hypodermic needles being in fluid communication with a hemodialysis machine;
circulating blood through the hemodialysis machine; and
after a sufficient amount of blood has been circulated through the hemodialysis machine, closing the first and second valve devices using the actuator.

10. A method as in claim 9, wherein both the arterial end and the venous end each comprise a support device.

11. A method as in claim 9, wherein the support frame comprises a stent.

12. A method as in claim 9, wherein the support frame is encapsulated within the graft.

13. A method as in claim 9, wherein each valve device exerts a pressure of between about 20 psi and about 40 psi against the support frame when closing the valve devices.

14. A method as in claim 9, wherein the arteriovenous graft system further comprises a coupling insert, the coupling insert joining the arteriovenous graft to the support frame.

15. A method as in claim 14, wherein the arteriovenous graft and the support frame define a flow path, the coupling insert being positioned within the flow path.

16. A method as in claim 14, wherein the arteriovenous graft and the support frame define a flow path, the support frame assisting in reopening the flow path when the valve device is opened by the actuator.
